(19) United States
(12) Patent Application Publication
(10) Pub. No.: US 2019/0151340 A1
(43) Pub. Date: May 23, 2019

(54) POLYMERIC NANOPARTICLES COMPRISING BORTEZOMIB

(71) Applicant: HILLSTREAM BIOPHARMA INC., CHESTER, NJ (US)

(72) Inventors: HARPAL SINGH, CHESTER, NJ (US); SURENDER KHARBANDA, CHESTER, NJ (US)

(21) Appl. No.: 16/196,130

(22) Filed: Nov. 20, 2018

Related U.S. Application Data
(60) Provisional application No. 62/590,226, filed on Nov. 22, 2017.

(51) Int. Cl. A61K 31/69; A61K 9/51; A61K 9/00; A61K 47/10; A61K 47/34; A61K 45/06; A61P 35/02

(52) U.S. Cl. CPC A61K 31/69; A61K 9/5153; A61K 9/0019; A61P 35/02; A61K 47/34; A61K 45/06; A61K 47/10

(57) ABSTRACT

The present invention relates to polymeric nanoparticles comprising bortezomib and methods for treating certain diseases comprising administering these polymeric nanoparticles to a subject in need thereof.

### RELATED APPLICATIONS

This application claims priority to U.S. Ser. No. 62/590,226, filed Nov. 22, 2017. The contents of this application are incorporated herein by reference in their entirety.

### FIELD

The present invention relates to the field of nanotechnology and more particularly to the use of biodegradable polymeric nanoparticles for the delivery of therapeutic agents such as bortezomib.

### BACKGROUND

Bortezomib (N-2-pyrazinecarbonyl-L-phenylalanine-L-leucineboronic acid), a boronated dipeptidic compound with L-leucine and L-phenylalanine moieties, is a selective proteasome inhibitor. Inhibition of proteasomes by bortezomib affects cancer cells in a number of ways, including causing cell cycle arrest and apoptosis. The compound has been given regulatory approval for treating multiple myeloma, including relapsed multiple myeloma, and certain lymphomas, including mantle cell lymphoma. Other potential uses of bortezomib also have been reported, including treatment of amyloidosis.

### SUMMARY

The disclosure is based in part on the discovery that nanoparticles comprising bortezomib are more effective than bortezomib alone in treating multiple myeloma (MM). Accordingly, in one aspect, the invention provides a composition comprising: polymeric nanoparticles comprising a block copolymer comprising poly(lactic acid) (PLA) and poly(ethylene glycol) (PEG); and bortezomib.

The disclosure provides a composition comprises a polymeric nanoparticle comprises poly(lactic acid)-poly(ethylene glycol)-poly(propylene glycol)-poly(ethylene glycol) (PLA-PEG-PPG-PEG) tetra-block copolymer and bortezomib.

In various embodiments of the composition, the PLA-PEG-PPG-PEG tetra-block copolymer is formed from conjugation of PEG-PPG-PEG tri-block copolymer with PLA. For example, the conjugation is a chemical conjugation.

In various embodiments of the composition, the molecular weight of PLA is between about 10,000 and about 100,000 Daltons; between about 20,000 and 90,000 Daltons; between about 30,000 and 80,000 Daltons; between about 8,000 Daltons and 18,000 Daltons; or between about 10,000 Daltons and 15,000. For example, the molecular weight of the PLA is about 10,000; 20,000; 30,000; 40,000; 50,000; 60,000; 70,000; 80,000; 90,000, or 100,000 Daltons. In a further embodiment, the molecular weight of the PLA is about 12,500 Daltons (i.e., 12.5 kDA) or about 72,000 Daltons (i.e., 72 kDA). In an embodiment, the molecular weight of PEG-PPG-PEG for generating the tetra block in an A-B structure, i.e., an alternating copolymer with regular alternating A and B subunits, is 12.5 kDa. In various embodiments, the composition further comprises a chemotherapeutic agent or a targeted anti-cancer agent selected from the group consisting of lenalidomide, crizotinib, gleevec, herceptin, avastin, PD-1 checkpoint inhibitors, PDL-1 checkpoint inhibitors, and CTLA-4 checkpoint inhibitors and combinations thereof.

In various embodiments of the composition, the polymeric nanoparticles are formed of a polymer consisting essentially of poly(lactic acid)-poly(ethylene glycol) (PLA-PEG) di-block copolymer.

In various embodiments of the composition, the polymeric nanoparticles are formed of a polymer consisting essentially of poly(lactic acid)-poly(ethylene glycol)-poly(propylene glycol)-poly(ethylene glycol) (PLA-PEG-PPG-PEG) tetra-block copolymer.

In various embodiments of the composition, the polymeric nanoparticles further comprise a targeting moiety attached to the outside of the polymeric nanoparticles, and wherein the targeting moiety is an antibody, peptide, or aptamer. In various embodiments the targeting moiety comprises an immunoglobulin molecule, an scFv, a monoclonal antibody, a humanized antibody, a chimeric antibody, a humanized antibody, a Fab fragment, an Fab′ fragment, an F(ab′)2, an Fv, and a disulfide linked Fv.

In various embodiments of any of the compositions or methods provided herein, the nanoparticle is formed of the block copolymer comprising poly(lactic acid) (PLA) and poly(ethylene glycol) (PEG); and bortezomib. In an embodiment, the nanoparticle releases bortezomib over a period of time. In a further embodiment, the period of time is at least 1 day to 20 days. In various embodiments of the method, the period of time is about 5 days to 10 days.

The disclosure also provides a pharmaceutical composition comprising a polymeric nanoparticle comprises poly(lactic acid)-poly(ethylene glycol)-poly(propylene glycol)-poly(ethylene glycol) (PLA-PEG-PPG-PEG) tetra-block copolymer, bortezomib and a pharmaceutically acceptable carrier. In certain embodiments, the polymeric nanoparticle further comprises a targeting moiety attached to the outside of the polymeric nanoparticles.

The disclosure also provides a method of treating a cell exhibiting symptoms of cancer comprising contacting the cell with a therapeutically effective amount of a composition comprising a polymeric nanoparticle comprises poly(lactic acid)-poly(ethylene glycol)-poly(propylene glycol)-poly(ethylene glycol) (PLA-PEG-PPG-PEG) tetra-block copolymer and bortezomib. In certain embodiments, the cell is one or more of a cell from a subject or a cultured cell. In specific embodiments, the cell from the subject is one or more of bone marrow stromal cell (BMSC), a peripheral blood mononuclear cell (PBMC), lymphocytes, hair follicles, blood cells, other epithelial cells, bone marrow plasma cells, primary cancer cells, patient derived tumor cells, normal or cancerous hematopoietic stem cells, neural stem cells, solid tumor cells, or astrocytes.

The disclosure also provides a method for treating a subject at risk for or having a hematological malignancy or disorder associated with same, the method comprising administering to a subject in need thereof a therapeutically effective amount a composition comprising a polymeric nanoparticle comprises poly(lactic acid)-poly(ethylene glycol)-poly(propylene glycol)-poly(ethylene glycol) (PLA-PEG-PPG-PEG) tetra-block copolymer and bortezomib and a pharmaceutically effective carrier.

In certain embodiments, the hematological malignancy or disorder is multiple myeloma (MM) or lymphoma. In other embodiments, the hematological malignancy is myelodysplastic syndrome, Hodgkin's lymphoma, chronic lymphocytic leukemia, acute myelogenous leukemia or B cell lymphoma. In other embodiments, the subject is at risk for monoclonal Gammopathy of Undetermined Significance (MGUS), smoldering myeloma, asymptomatic MM, or symptomatic MM. Optionally, the symptomatic MM is newly diagnosed MM or late stage relapsed/refractory MM.

In certain embodiments, the method also includes administering an additional anti-cancer therapy to the subject. In certain embodiments, the additional anti-cancer therapy is surgery, chemotherapy, radiation, hormone therapy, immunotherapy, or a combination thereof. Optionally, the additional anti-cancer therapy reduces bone absorption or reduces osteoclast mediated bone resorption. In certain embodiments, the additional anti-cancer therapy is a bisphosphonate. In other embodiments, the subject is a human.

In certain embodiments, administration is via a route selected from the group consisting of subcutaneous, intravenous, and intraperitoneal delivery. In another embodiment, administration of the composition does not induce weight loss in the subject.

The disclosure also provides a method of reducing proliferation, survival, migration, or colony formation ability of multiple myeloma cells in a subject with multiple myeloma, the method comprising administering to the subject a therapeutically effective amount of a composition comprising a polymeric nanoparticle comprises poly(lactic acid)-poly(ethylene glycol)-poly(propylene glycol)-poly(ethylene glycol) (PLA-PEG-PPG-PEG) tetra-block copolymer and bortezomib and a pharmaceutically effective carrier.

In certain embodiments, the hematological malignancy or disorder is multiple myeloma (MM) or lymphoma. In other embodiments, the hematological malignancy is myelodysplastic syndrome, Hodgkin's lymphoma, chronic lymphocytic leukemia, or B cell lymphoma. In other embodiments, the subject is at risk for monoclonal Gammopathy of Undetermined Significance (MGUS), smoldering myeloma, asymptomatic MM, or symptomatic MM. Optionally, the symptomatic MM is newly diagnosed MM or late stage relapsed/refractory MM. In certain embodiments, administration is via a route selected from the group consisting of subcutaneous, intravenous, and intraperitoneal delivery. In certain embodiments, the method also includes administering an additional anti-cancer therapy to the subject. In certain embodiments, the additional anti-cancer therapy is surgery, chemotherapy, radiation, hormone therapy, immunotherapy, or a combination thereof. In certain embodiments, the additional anti-cancer therapy reduces bone absorption. In other embodiments, the additional anti-cancer therapy reduces osteoclast mediated bone resorption. In certain embodiments, the additional anti-cancer therapy is a bisphosphonate. In certain embodiments, the subject is a human. In certain embodiments, administration is via a route selected from the group consisting of subcutaneous, intravenous, and intraperitoneal delivery.

The disclosure also provides a method of reducing proliferation, survival, migration, or colony formation ability of multiple myeloma cells in a subject with multiple myeloma, the method comprising administering to the subject a therapeutically effective amount of a composition comprising a polymeric nanoparticle comprises poly(lactic acid)-poly(ethylene glycol)-poly(propylene glycol)-poly(ethylene glycol) (PLA-PEG-PPG-PEG) tetra-block copolymer and bortezomib and a pharmaceutically effective carrier. In certain embodiments, administration is via a route selected from the group consisting of subcutaneous, intravenous, and intraperitoneal delivery.

The disclosure also provides a method of inhibiting metastasis of myeloma in a subject, the method comprising administering to a subject with myeloma a therapeutically effective amount of a composition comprising a polymeric nanoparticle comprises poly(lactic acid)-poly(ethylene glycol)-poly(propylene glycol)-poly(ethylene glycol) (PLA-PEG-PPG-PEG) tetra-block copolymer and bortezomib and a pharmaceutically effective carrier. In certain embodiments, administration is via a route selected from the group consisting of subcutaneous, intravenous, and intraperitoneal delivery.

In various embodiments of the method, the cancer is a hematological cancer or related condition.

In various embodiments of the method, the cancer is breast cancer, prostate cancer, non-small cell lung cancer, metastatic colon cancer, pancreatic cancer, or a malignancy. For example, the cancer comprises a PD-1 refractory tumor.

Those skilled in the art will be aware that the invention described herein is subject to variations and modifications other than those specifically described. It is to be understood that the invention described herein includes all such variations and modifications. The invention also includes all such steps, features, compositions and compounds referred to or indicated in this specification, individually or collectively, and any and all combinations of any two or more of the steps or features.

### BRIEF DESCRIPTION OF THE FIGURES

The following figures form part of the present specification and are included to further illustrate aspects of the present invention.

FIGS. 1A and 1B are graphs showing percent survival of RPMI-8226 Multiple Myeloma Cells at increasing concentrations (FIG. 1A) or doses (FIG. 1B) of bortezomib-containing nanoparticles.

FIGS. 2A and 2B are graphs showing percent survival of OPM-2 Multiple Myeloma Cells at increasing concentrations (FIG. 2A) or doses (FIG. 2B) of bortezomib-containing nanoparticles.

FIG. 3 is a graph showing tumor volume over time of in implanted RPMI-8226 MM animal xenografts treated with bortezomib-containing nanoparticles (circles) or vehicle (squares).

FIG. 4 is a graph showing body weight over time of MM RPMI-8226 xenograft mice treated with bortezomib-containing nanoparticles or vehicle.

FIG. 5 is a graph showing changes in body weight over time in wild-type mice treated with 1.5 mg/kg bortezomib alone or a bortezomib-containing nanoparticle.

FIG. 6 is a graph showing changes in body weight over time in wild-type mice treated with 3.0 mg/kg bortezomib alone or a bortezomib-containing nanoparticle.

FIG. 7 is a graph showing changes in body weight over time in wild-type mice treated with 6.0 mg/kg bortezomib alone or a bortezomib-containing nanoparticle.

FIG. 8 is a graph showing changes in body weight over time in wild-type mice treated with 9.0 mg/kg bortezomib alone or a bortezomib-containing nanoparticle.

FIG. 9 is a graph showing changes in body weight over time in wild-type mice treated with 12.0 mg/kg bortezomib alone or a bortezomib-containing nanoparticle.

FIGS. 10A and 10B are transmission electron micrographs of bortezomib tetra block polymeric nanoparticles.

FIG. 11 is a graph showing slow and sustained release of bortezomib in vitro in a cell free buffer system.

FIG. 12 is a graph showing percent proliferation of a MCF-7 hormone dependent breast cancer cell line when exposed to various concentrations of bortezomib (blue, bottom line) and bortezomib nanoparticle (red, top line).

FIG. 13 is a graph showing tumor volume (mm³) over time of RPMI-8226 multiple myeloma cells grown as s.c. xenograft in nu/nu mice.

### DETAILED DESCRIPTION

Provided are nanoparticles comprising bortezomib (product name VELCADE®) that are useful, inter alia, for treating or preventing cancers, including hematological cancers. Hematological cancers include, e.g., multiple myeloma and lymphoma and their associated conditions.

### Definitions

For convenience, before further description of the present invention, certain terms used in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art. The terms used throughout this specification are defined as follows, unless otherwise limited in specific instances.

The articles “a,” “an” and “the” are used to refer to one or to more than one (i.e., to at least one) of the grammatical object of the article.

By “anti-cancer therapy” is meant any treatment that slows the growth or metastasis of a tumor or a metastasis of a tumor.”

By “absorption” is meant the process of absorbing something (e.g., the anti-cancer therapy) or of being absorbed.

By “immunotherapy” is therapy that uses substances to stimulate or suppress the immune system to help the body fight cancer, infection, and other diseases. Some types of immunotherapy only target certain cells of the immune system. Others affect the immune system in a general way. Types of immunotherapy include cytokines, vaccines, bacillus Calmette-Guerin (BCG), and some monoclonal antibodies.

The terms “comprise”, “comprising”, “including” “containing”, “characterized by”, and grammatical equivalents thereof are used in the inclusive, open sense, meaning that additional elements may be included. It is not intended to be construed as “consists of only.”

As used herein, “consisting of” and grammatical equivalent thereof exclude any element, step or ingredient not specified in the claim.

As used herein, the term “about” or “approximately” usually means within 20%, more preferably within 10%, and most preferably still within 5% of a given value or range.

The term “biodegradable” as used herein refers to both enzymatic and non-enzymatic breakdown or degradation of the polymeric structure.

The term “cationic” refers to any agent, composition, molecule or material that has a net positive charge or positive zeta potential under the respective environmental conditions.

In various embodiments, nanoparticles described herein include a cationic polymer, peptide, protein carrier, or lipid.

By “hormone therapy” is meant treatment that adds, blocks, or removes hormones. By “immunotherapy” is meant therapy that uses substances to stimulate or suppress the immune system to help the body fight cancer, infection, and other diseases. Some types of immunotherapy only target certain cells of the immune system. Others affect the immune system in a general way. Types of immunotherapy include cytokines, vaccines, bacillus Calmette-Guerin (BCG), and some monoclonal antibodies.

By “osteoclast” is meant a bone cell that is large, multinucleated, and associated with bone resorption.

By “resorption of bone tissue” is meant the process by which osteoclasts break down the tissue in bones and release the minerals, resulting in a transfer of calcium from bone tissue to the blood.

By “lymphoma” it is meant a malignant growth of B or T cells in the lymphatic system, optionally including Hodgkin's lymphoma or non-Hodgkin's lymphoma (NHL). In embodiments, the non-Hodgkin's Lymphoma is a selected from the group consisting of aggressive NHL, transformed NHL, indolent NHL, relapsed NHL, refractory NHL, low grade non-Hodgkin's Lymphoma, follicular lymphoma, large cell lymphoma, B-cell lymphoma, T-cell lymphoma, mantle cell lymphoma, Burkitt's lymphoma. NK cell lymphoma, diffuse large B-cell lymphoma, acute lymphoblastic lymphoma, and cutaneous T cell cancer, including mycosos fungoides/Sezry syndrome. An “indolent” non-Hodgkin's Lymphoma is a classification that includes slow growing forms of lymphoma. They encompass what are called low grade and some categories of intermediate grade NHL in the Working Formulation. Indolent NHLs are sometimes not responsive to conventional cancer therapies such as chemotherapy and radiation therapy. Indolent NHL and other premalignant forms of NHL may also proceed to NHL. With regard to premalignant or benign forms of the disease, optionally the compositions and methods thereof may be applied for prevention, in addition to or in place of treatment, for example optionally to halt the progression of the disease to a malignant form of NHL. A “transformed” non-Hodgkin's Lymphoma is a classification sometimes employed to describe an indolent NHL which acquires an aggressive aspect and becomes more responsive to standard chemotherapies.

By “multiple myeloma” it is meant any type of B-cell malignancy characterized by the accumulation of terminally differentiated B-cells (plasma cells) in the bone marrow. The multiple myeloma cancer can be one of several that produce light chains of kappa-type and/or light chains of lambda-type; and/or aggressive multiple myeloma, including primary plasma cell leukemia (PCL); and/or optionally including benign plasma cell disorders such as MGUS (monoclonal gammopathy of undetermined significance) and/or Waldenstrom's macroglobulinemia (WM, also known as lymphoplasmacytic lymphoma) which may proceed to multiple myeloma; and/or smoldering multiple myeloma (SMM), and/or indolent multiple myeloma, and/or retreatment of multiple myeloma, premalignant forms of multiple myeloma which may also proceed to multiple myeloma; and/or primary amyloidosis. With regard to premalignant or benign forms of the disease, optionally the compositions and methods thereof may be applied for prevention, in addition to or in place of treatment, for example optionally to halt the progression of the disease to a malignant form of multiple myeloma.

As used herein, “refractory myeloma” is disease that is progressing despite active treatment. Refractory multiple myeloma can include two types of patients: 1. Primary refractory patients who never achieve a response and progress while still on induction therapy (including chemotherapy), or their myeloma never responded to treatment initially). 2. Secondary refractory patients who do respond to induction chemotherapy but do not respond to treatment after relapse. This includes situations in which myeloma medications worked initially but no longer work after relapse of disease relapsed, they no longer work.

As used herein, the term “nanoparticle” refers to particles in the range between 10 nm to 1000 nm in diameter, wherein diameter refers to the diameter of a perfect sphere having the same volume as the particle. The term “nanoparticle” is used interchangeably as “nanoparticle(s)”. In some cases, the diameter of the particle is in the range of about 1-1000 nm, 10-500 nm, 20-300 nm, or 100-300 nm. In various embodiments, the diameter is about 30-170 nm. In embodiments, the diameter of the nanoparticle is 1, 5, 10, 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1000 nm.

In some cases, a population of particles may be present. As used herein, the diameter of the nanoparticles is an average of a distribution in a particular population.

As used herein, the term “polymer” is given its ordinary meaning as used in the art, i.e., a molecular structure comprising one or more repeat units (monomers), connected by covalent bonds. The repeat units may all be identical, or in some cases, there may be more than one type of repeat unit present within the polymer.

As used herein, the terms “chemotherapeutic agent”, “therapeutic agent” and “drug” are used interchangeably and are also intended to encompass not only compounds or species that are inherently pharmaceutically or biologically active, but materials which include one or more of these active compounds or species, as well as conjugations, modification, and pharmacologically active fragments, and antibody derivatives thereof.

A “targeting moiety” is a molecule that will bind selectively to the surface of targeted cells. For example, the targeting moiety may be a ligand that binds to the cell surface receptor found on a particular type of cell or expressed at a higher frequency on target cells than on other cells.

The targeting moiety or therapeutic agent can be a peptide or protein. “Proteins” and “peptides” are well-known terms in the art, and as used herein, these terms are given their ordinary meaning in the art. Generally, peptides are amino acid sequences of less than about 100 amino acids in length, but can include up to 300 amino acids. Proteins are generally considered to be molecules of at least 100 amino acids. The amino acids can be in D- or L-configuration. A protein can be, for example, a protein drug, an antibody, a recombinant antibody, a recombinant protein, an enzyme, or the like. In some cases, one or more of the amino acids of the peptide or protein can be modified, for example by the addition of a chemical entity such as a carbohydrate group, a phosphate group, a farnesyl group, an isofarnesyl group, a fatty acid group, a linker for conjugation, functionalization, or other modification such as cyclization, by-cyclization and any of numerous other modifications intended to confer more advantageous properties on peptides and proteins. In other instances one or more of the amino acids of the peptide or protein can be modified by substitution with one or more non-naturally occurring amino acids. The peptides or proteins may by selected from a combinatorial library such as a phage library, a yeast library, or an in vitro combinatorial library.

The term “combination,” “therapeutic combination,” or “pharmaceutical combination” as used herein refer to the combined administration of two or more therapeutic agents (e.g., co-delivery). Components of a combination therapy may be administered simultaneously or sequentially, i.e., at least one component of the combination is administered at a time temporally distinct from the other component(s). In embodiments, a component(s) is administered within one month, one week, 1-6 days, 18, 12, 10, 9, 8, 7, 6, 5, 4, 3, 2, 1 hour, or 30, 20, 15, 10, or 5 minutes of the other component(s).

The term “pharmaceutically acceptable” as used herein refers to those compounds, materials, compositions and/or dosage forms, which are, within the scope of sound medical judgment, suitable for contact with the tissues a warm-blooded animal, e.g., a mammal or human, without excessive toxicity, irritation allergic response and other problem complications commensurate with a reasonable benefit/risk ratio.

A “therapeutically effective amount” of a polymeric nanoparticle comprising one or more therapeutic agents is an amount sufficient to provide an observable or clinically significant improvement over the baseline clinically observable signs and symptoms of the disorders treated with the combination.

The term “subject” or “patient” as used herein is intended to include animals, which are capable of suffering from or afflicted with a cancer or any disorder involving, directly or indirectly, a cancer. Examples of subjects include mammals, e.g., humans, apes, monkeys, dogs, cows, horses, pigs, sheep, goats, cats, mice, rabbits, rats, and transgenic non-human animals. In an embodiment, the subject is a human, e.g., a human suffering from, at risk of suffering from, or potentially capable of suffering from cancers.

The term “treating” or “treatment” as used herein comprises a treatment relieving, reducing or alleviating at least one symptom in a subject or producing a delay in the progression of a disease. For example, treatment can be the diminishment of one or several symptoms of a disorder or complete eradication of a disorder, such as cancer. Within the meaning of the present disclosure, the term “treat” also denotes to arrest and/or reduce the risk of worsening a disease. The term “prevent”, “preventing” or “prevention” as used herein comprises the prevention of at least one symptom associated with or caused by the state, disease or disorder being prevented.

As used herein, the term “hematological disorder: means a disease or condition manifested by a cancerous or precancerous state of a cell found in blood cancer that begins in blood-forming tissue, such as the bone marrow, or in the cells of the immune system. Examples include multiple myeloma, leukemia, lymphoma (also called blood cancer) and their associated diseases or conditions. Additional diseases or conditions can include, e.g., leukemia, e.g., acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

In some embodiments, the methods disclosed herein may be used in order to stage or restage the disease in individuals having a recurrent or relapsed multiple myeloma, i.e. a multiple myeloma that returns after a period of being in control, e.g. after a therapeutic treatment.

In the context of relapsed and/or refractory, three groups of patients exist. The first is a group that has “relapsed” disease, which specifically includes patients whose first progression occurs in the absence of any therapy following successful initial therapy. Although the definition of relapsed disease requires a ≥25% increase in the serum or urine protein and ≥0.5 mg/dL, the presence of “biochemical” relapse alone is not indication for additional systemic therapy. Because the patient time to relapse can be quite variable (weeks to months), patients should have some form of symptomatic relapse prior to initiation of therapy, because many patients could survive for some time with biochemical progression and yet not require additional therapy beyond careful monitoring. The next category includes patients having relapsed and refractory disease who are defined as progressing on a specific therapy, or within 60 days of completion of a given therapy (International Myeloma Working Group Consensus Panel, International Myeloma Workshop, February 2009).

Historically, this was limited to steroid or alkylator-based approaches; thus, “refractory” was a generic term. But, more recently, it has become associated with specific agents, such as bortezomib or lenalidomide refractory relapse. This is clearly important because patients who are refractory to bortezomib may still be responsive to lenalidomide or vice versa, and this agent-specific resistance may continue to be relevant for the sequential evaluation and integration of new agents that are in development in the relapsed setting. This group of patients may be especially challenging among the group of patients who have received multiple prior lines of therapy and outside of clinical trials have few treatment options.

The final category is primary refractory, which also represents a potentially challenging group of patients who did not achieve a response following induction therapy. As with refractory disease, this category is most useful when described in the context of specific agents or combinations, and it is particularly important to distinguish the group of patients who can have a variable course with less aggressive tempo of disease despite initial resistance.

### Polymeric Nanoparticles Comprising Bortezomib

Bortezomib is well known in the art and disclosed in, e.g., U.S. Pat. No. 6,713,446, Albanell and Adams, Drugs of the Future 27: 1079-1092 (2002), which reports that bortezomib (N-2-pyrazinecarbonyl-L-phenylalanine-L-leucineboronic acid) shows significant antitumor activity in human tumor xenograft models. See also Richardson et al., New Engl. J. Med., 348:2609 (2003), which report the results of a Phase 2 study of bortezomib, showing its effectiveness in treating relapsed and refractory multiple myeloma.

Provided herein are biodegradable polymeric nanoparticles for the delivery of bortezomib. Nanoparticles comprising bortezomib can be prepared using methods described in, e.g., US 2015-0353676 A1; PCT/US2016/060276 (published May 11, 2017); and PCT/US2017/059542, filed Nov. 1, 2017, published May 11, 2018.

In an embodiment, the polymeric nanoparticles provided herein comprise a block copolymer comprising poly(lactic acid) (PLA) and poly(ethylene glycol) (PEG). Poly(lactic acid) (PLA), is a hydrophobic polymer, and is a preferred polymer for synthesis of the polymeric nanoparticles. However, poly(glycolic acid) (PGA) and block copolymer of poly lactic acid-co-glycolic acid (PLGA) may also be used. The hydrophobic polymer can also be biologically derived or a biopolymer. The molecular weight of the PLA used is generally in the range of about 2,000 g/mol to 80,000 g/mol. Thus, in an embodiment, the PLA used is in the range of about 10,000 g/mol to 80,000 g/mol. The average molecular weight of PLA may also be about 70,000 g/mol.

PEG is another preferred component to of the polymer used to form the polymeric nanoparticles as it imparts hydrophilicity, anti-phagocytosis against macrophage, and resistance to immunological recognition. Block copolymers like poly(ethylene glycol)-poly(propylene glycol)-poly(ethylene glycol) (PEG-PPG-PEG) are hydrophilic or hydrophilic-hydrophobic copolymers that can be used in the present invention. Block copolymers may have two, three, four, or more numbers of distinct blocks.

As used herein, one g/mole is equivalent to one “Dalton” (i.e., Dalton and g/mol are interchangeable when referring to the molecular weight of a polymer). “Kilodalton” as used herein refers to 1,000 Daltons.

In a further embodiment, the polymeric nanoparticles provided herein comprise poly(lactic acid)-poly(ethylene glycol) (PLA-PEG) di-block copolymer.

In yet a further embodiment, the polymeric nanoparticles provided herein comprise poly(lactic acid)-poly(ethylene glycol)-poly(propylene glycol)-poly(ethylene glycol) (PLA-PEG-PPG-PEG) tetra-block copolymer. In various embodiments, the nanoparticles comprise a NANOPRO™, which is a biodegradable, long blood circulating, stealth, tetra-block polymeric nanoparticle platform (NanoProteagen Inc.; Massachusetts). The PLA-PEG-PPG-PEG tetra-block copolymer can be formed from chemical conjugation of PEG-PPG-PEG tri-block copolymer with PLA.

The synthesis and characterization of nanoparticles comprising poly(lactic acid)-poly(ethylene glycol)-poly(propylene glycol)-poly(ethylene glycol) (PLA-PEG-PPG-PEG) tetra block copolymer are described in PCT publication no. WO2013/160773, which is hereby incorporated by reference in its entirety. Polymeric nanoparticles comprising poly(lactic acid)-poly(ethylene glycol)-poly(propylene glycol)-poly(ethylene glycol) (PLA-PEG-PPG-PEG) tetra block copolymer have been shown to be safe, stable and non-toxic.

The process used to form this tetra-block copolymer comprises covalently attaching PEG-PPG-PEG to the poly-lactic acid (PLA) matrix, resulting in the block copolymer becoming a part of the matrix, i.e., a nanoparticle delivery system. This prevents leaching out of emulsifier into the medium.

In some embodiments, the average molecular weight (Mn) of the hydrophilic-hydrophobic block copolymer (e.g., PEG-PPG-PEG) is generally in the range of 1,000 to 20,000 g/mol. In a further embodiment, the average molecular weight (Mn) of the hydrophilic-hydrophobic block copolymer is about 4,000 g/mol to 15,000 g/mol. In some cases, the average molecular weight (Mn) of the hydrophilic-hydrophobic block copolymer is 4,400 g/mol, 8,400 g/mol, or 14,600 g/mol. In certain embodiments, the Mn of PEG-PPG-PEG is 1,100-15,000 g/mol, e.g., 4,000 to 13,000 g/mol. In certain embodiments, the Mn of PEG-PPG-PEG is 10,000-13,000 g/mol. In other embodiments, the Mn of PEG-PPG-PEG is about 12,500 g/mol.

In some embodiments, a block copolymer of the instant invention consists essentially of a segment of poly(lactic acid) (PLA) and a segment of poly(ethylene glycol)-poly(propylene glycol)-poly(ethylene glycol) (PEG-PPG-PEG).

In an embodiment, a specific biodegradable polymeric nanoparticle is formed of the block copolymer poly(lactic acid)-poly(ethylene glycol)-poly(propylene glycol)-poly(ethylene glycol) (PLA-PEG-PPG-PEG).

Another specific biodegradable polymeric nanoparticle of the instant invention is formed of the block copolymer poly(lactic acid)-poly(ethylene glycol)-poly(propylene glycol)-poly(ethylene glycol)-poly(lactic acid) (PLA-PEG-PPG-PEG-PLA).

In embodiments, a bortezomib-comprising nanoparticle does not include a NuBCP-9 peptide, a MUC-1 peptide, and/or tumor necrosis factor alpha (TNFα).

The biodegradable polymers of the instant invention can be formed by chemically modifying PLA with a hydrophilic-hydrophobic block copolymer using a covalent bond.

The biodegradable polymeric nanoparticles of the instant invention have, in various embodiments, a size in the range of about 1-1000 nm, a size in the range of about 30-300 nm, a size in the range of about 100-300 nm, or a size in the range of about 100-250 nm, or a size of at least about 100 nm.

The biodegradable polymeric nanoparticles of the instant invention have, in various embodiments, a size in the range of about 30-120 nm, a size of about 120-200 nm, or a size of about 200-260 nm, or a size of at least about 260 nm.

In an embodiment, the biodegradable polymer of the instant invention is substantially free of emulsifier, or may comprise external emulsifier by an amount of about 0.5% to 5% by weight.

In an embodiment, the biodegradable polymeric nanoparticle of the present invention is PLA-PEG-PPG-PEG, and the average molecular weight of the poly(lactic acid) block is about 60,000 g/mol, the average weight of the PEG-PPG-PEG block is about 8,400 or about 14,600 g/mol, and the external emulsifier is about 0.5% to 5% by weight.

In another embodiment, the biodegradable polymeric nanoparticle of the present invention is PLA-PEG-PPG-PEG, and the an average molecular weight of the poly(lactic acid) block is less than or equal to approximately 16,000 g/mol, the average weight of the PEG-PPG-PEG block is about 8,400 g/mol or about 14,600 g/mol, and wherein the composition is substantially free of emulsifier.

In an embodiment, the biodegradable polymeric nanoparticle is PLA-PEG-PPG-PEG, and the average molecular weight of the poly(lactic acid) block is about 72,000 g/mol (or 72 kDa), the average weight of the PEG-PPG-PEG block is about 8,400 or about 14,600 g/mol, and the external emulsifier is about 0.5% to 5% by weight.

In another embodiment, the biodegradable polymeric nanoparticle is PLA-PEG-PPG-PEG, and the an average molecular weight of the poly(lactic acid) block is less than or equal to approximately 12,000 g/mol (or 12 kDa), the average weight of the PEG-PPG-PEG block is about 8,400 g/mol or about 14,600 g/mol, and wherein the composition is substantially free of emulsifier.

In another embodiment, the polymeric nanoparticles provided herein further comprise a cationic peptide.

In another aspect, provided herein is a polymeric nanoparticle formed of a polymer consisting essentially of a PLA-PEG-PPG-PEG tetra-block copolymer or PLA-PEG di-block copolymer, wherein the polymeric nanoparticles are loaded with bortezomib and, optionally, a second therapeutic agent.

Nanoparticles (also referred to herein as “NPs”) can be produced as nanocapsules or nanospheres. Bortezomib loading in the nanoparticle can be performed by either an adsorption process or an encapsulation process (Spada et al., 2011; Protein delivery of polymeric nanoparticles; World Academy of Science, Engineering and Technology: 76). Nanoparticles, by using both passive and active targeting strategies, can enhance the intracellular concentration of drugs in cancer cells while avoiding toxicity in normal cells. When nanoparticles bind to specific receptors and enter the cell, they are usually enveloped by endosomes via receptor-mediated endocytosis, thereby bypassing the recognition of P-glycoprotein, one of the main drug resistance mechanisms (Cho et al., 2008, Therapeutic Nanoparticles for Drug Delivery in Cancer, Clin. Cancer Res., 2008, 14:1310-1316). Nanoparticles are removed from the body by opsonization and phagocytosis (Sosnik et al., 2008; Polymeric Nanocarriers: New Endeavors for the Optimization of the Technological Aspects of Drugs; Recent Patents on Biomedical Engineering, 1: 43-59). Nanocarrier based systems can be used for effective drug delivery with the advantages of improved intracellular penetration, localized delivery, protect drugs against premature degradation, controlled pharmacokinetic and drug tissue distribution profile, lower dose requirement and cost effectiveness (Farokhzad O C, et al.; Targeted nanoparticle-aptamer bioconjugates for cancer chemotherapy in vivo. Proc. Natl. Acad. Sci. USA 2006, 103 (16): 6315-20; Fonseca C, et al., Paclitaxel-loaded PLGA nanoparticles: preparation, physicochemical characterization and in vitro anti-tumoral activity. J. Controlled Release 2002; 83 (2): 273-86; Hood et al., Nanomedicine, 2011, 6(7):1257-1272).

The uptake of nanoparticles is indirectly proportional to their small dimensions. Due to their small size, the polymeric nanoparticles have been found to evade recognition and uptake by the reticulo-endothelial system (RES), and can thus circulate in the blood for an extended period (Borchard et al., 1996, Pharm. Res. 7: 1055-1058). Nanoparticles are also able to extravasate at the pathological site like the leaky vasculature of a solid tumor, providing a passive targeting mechanism. Due to the higher surface area leading to faster solubilization rates, nano-sized structures usually show higher plasma concentrations and area under the curve (AUC) values. Lower particle size helps in evading the host defense mechanism and increase the blood circulation time. Nanoparticle size affects drug release. Larger particles have slower diffusion of drugs into the system. Smaller particles offer larger surface area but lead to fast drug release. Smaller particles tend to aggregate during storage and transportation of nanoparticle dispersions. Hence, a compromise between a small size and maximum stability of nanoparticles is desired. The size of nanoparticles used in a drug delivery system should be large enough to prevent their rapid leakage into blood capillaries but small enough to escape capture by fixed macrophages that are lodged in the reticuloendothelial system, such as the liver and spleen.

In addition to their size, the surface characteristics of nanoparticles are also an important factor in determining the life span and fate during circulation. Nanoparticles should ideally have a hydrophilic surface to escape macrophage capture. Nanoparticles formed from block copolymers with hydrophilic and hydrophobic domains meet these criteria. Controlled polymer degradation also allows for increased levels of agent delivery to a diseased state. Polymer degradation can also be affected by the particle size. Degradation rates increase with increase in particle size in vitro (Biopolymeric nanoparticles; Sundar et al., 2010, Science and Technology of Advanced Materials; doi:10.1088/1468-6996/11/1/014104). Poly(lactic acid) (PLA) has been approved by the US FDA for applications in tissue engineering, medical materials and drug carriers and poly(lactic acid)-poly(ethylene glycol) PLA-PEG based drug delivery systems are known in the art. US2006/0165987A1 describes a stealthy polymeric biodegradable nanosphere comprising poly(ester)-poly(ethylene) multiblock copolymers and optional components for imparting rigidity to the nanospheres and incorporating pharmaceutical compounds. US2008/0081075A1 discloses a novel mixed micelle structure with a functional inner core and hydrophilic outer shells, self-assembled from a graft macromolecule and one or more block copolymer. US2010/0004398A1 describes a polymeric nanoparticle of shell/core configuration with an interphase region and a process for producing the same.

In various embodiments, the invention further comprises a cationic molecule that interacts with a therapeutic molecule to form a stable nanocomplex and/or serves as a cell penetrating peptide. In various embodiments, the cationic molecule cell comprises a penetrating peptide comprises or a protein transduction domain. In various embodiments, the cationic molecule is a cationic peptide that facilitates transduction of the therapeutic agent to the nucleus.

Provided herein are methods for preparing a polymeric nanoparticle comprising borzone or more therapeutics. The resulting polymeric nanoparticle is not only non-toxic, safe, and biodegradable, but also stable in vivo with high storage stability, and can be safely used in a nanocarrier system or drug delivery system in the field of medicine. In embodiments, the polymeric nanoparticles provided herein can increase the half-life of the deliverable drug or therapeutic agent in-vivo

The preparation process can include providing bortezomib, dissolving a block polymer in a solvent to form a block copolymer solution; and adding the complex to the block copolymer solution to form a solution comprising the complex and the block copolymer.

In an embodiment, the block copolymer is PLA-PEG di-block copolymer.

In an embodiment, the block copolymer is PLA-PEG-PPG-PEG tetra-block copolymer.

In an embodiment, the block copolymer solution is prepared at a concentration between about 2 mg/ml and 10 mg/ml. In a further embodiment, the block copolymer solution of is prepared at a concentration of about 6 mg/ml.

In an embodiment, the process further comprises adding the solution comprising bortezomib to a solution comprising a surfactant. In a further embodiment, the solution resulting from combining bortezomib and the block polymer solution is stirred until stable nanoparticles are formed.

In various embodiments, the polymeric nanoparticles can adopt a non-spherical configuration upon swelling or shrinking.

The nanoparticle in various embodiments is amphiphilic in nature.

The zeta potential and PDI (Polydispersity Index) of the nanoparticles may be calculated (see U.S. Pat. No. 9,149,426).

The polymeric nanoparticles have dimensions that may be measured using a Transmission Electron Microscope. In suitable embodiments, the diameter of the polymeric nanoparticles provided herein will be between about 100 and 350 nm in diameter or between about 100 and 30 nm in diameter or between about 100 and 250 nm. In a further embodiment, the diameter of the polymeric nanoparticles provided herein are about 100 nm, 110 nm, 120, nm, 130 nm, 140 nm, 150 nm, 160 nm, 170 nm, 180 nm, 190 nm, 200 nm, 210 nm, 220 nm, 230 nm, 240 nm, or 250 nm.

In an embodiment, the polymeric nanoparticles comprising a complex have a zeta-potential between about +5 to −90 mV, e.g., +4 to −75 mV, +3 to −30 mV, +2 to −25 mV, +1 to −40 mV. In a further embodiment, the complex has a zeta-potential of about −30 mV.

Specific processes for polymeric nanoparticle formation and uses in pharmaceutical composition are provided herein for purpose of reference. These processes and uses may be carried out through a variety of methods apparent to those of skill in the art.

Pharmaceutical Compositions

Also provided herein is a pharmaceutical composition comprising a bortezomib polymeric nanoparticle for use in medicine and in other fields that use a carrier system or a reservoir or depot of nanoparticles. The nanoparticles can be used in prognostic, therapeutic, diagnostic and/or theranostic compositions. Suitably, the nanoparticles of the present invention are used for drug and agent delivery (e.g., within a tumor cell), as well as for disease diagnosis and medical imaging in human and animals. Thus, the instant invention provides a method for the treatment of disease using the nanoparticles further comprising a therapeutic agent as described herein. The nanoparticles of the present invention can also be use in other applications such as chemical or biological reactions where a reservoir or depot is required, as biosensors, as agents for immobilized enzymes and the like.

Thus, in an aspect, provided herein is a pharmaceutical composition comprising

a) a polymeric nanoparticle comprising a block copolymer comprising poly(lactic acid) (PLA) and poly(ethylene glycol) (PEG); and

b) bortezomib.

In an embodiment, the polymeric nanoparticle comprises poly(lactic acid)-poly(ethylene glycol) (PLA-PEG) di-block copolymer.

In an embodiment, the polymeric nanoparticle comprises poly(lactic acid)-poly(ethylene glycol)-poly(propylene glycol)-poly(ethylene glycol) (PLA-PEG-PPG-PEG) tetra-block copolymer.

In a further embodiment, the PLA-PEG-PPG-PEG tetra-block copolymer is formed from chemical conjugation of PEG-PPG-PEG tri-block copolymer with PLA.

In an embodiment, the molecular weight of PLA is between about 10,000 and about 100,000 Daltons.

In an embodiment of the compositions provided herein, the polymeric nanoparticles are formed of a polymer consisting essentially of poly(lactic acid)-poly(ethylene glycol) (PLA-PEG) di-block copolymer.

In an embodiment of the compositions provided herein, the polymeric nanoparticles are formed of a polymer consisting essentially of poly(lactic acid)-poly(ethylene glycol)-poly(propylene glycol)-poly(ethylene glycol) (PLA-PEG-PPG-PEG) tetra-block copolymer.

In an embodiment of the compositions provided herein, the polymeric nanoparticles further comprise a targeting moiety attached to the outside of the polymeric nanoparticles, and wherein the targeting moiety is an antibody, peptide, or aptamer.

Suitable pharmaceutical compositions or formulations can contain, for example, from about 0.1% to about 99.9%, preferably from about 1% to about 60%, of the active ingredient(s). Pharmaceutical formulations for enteral or parenteral administration are, for example, those in unit dosage forms, such as sugar-coated tablets, tablets, capsules or suppositories, or ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount may be reached by administration of a plurality of dosage units.

The pharmaceutical compositions can contain, as the active ingredient, one or more of nanoparticles in combination with one or more pharmaceutically acceptable carriers (excipients). In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

Some examples of suitable excipients include lactose (e.g. lactose monohydrate), dextrose, sucrose, sorbitol, mannitol, starches (e.g. sodium starch glycolate), gum acacia, calcium phosphate, alginates, tragacanth, gelatin, calcium silicate, colloidal silicon dioxide, microcrystalline cellulose, polyvinylpyrrolidone (e.g. povidone), cellulose, water, syrup, methyl cellulose, and hydroxypropyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Methods of Treatment

The nanoparticles disclosed herein can be used to treat or prevent any condition or disorder which is known to or suspected of benefitting from treatment with bortezomib, e.g., conditions or disorders for which selective inhibition of proteasomes is desired.

In one aspect, the bortezomib-containing nanoparticles are used to treat or prevent cancer or a precancerous condition. In embodiments the disease is a hematological disease. Examples of hematological diseases include, e.g., hematopoietic malignancies such as acute promyelocytic leukemia, T cell leukemia, acute lymphoblastic leukemia, Mantle cell lymphoma, B cell lymphoma, acute lymphoblastic T cell leukemia, neuroblastoma, adenocarcinoma, Ewing's sarcoma, glioblastoma, epithelial carcinoma, cervical adenocarcinoma, or well-differentiated liposarcoma cancers.

In embodiments, the condition treated includes multiple myeloma, lymphoma, or related conditions, e.g., Monoclonal Gammopathy of Undetermined Significance (MGUS), smoldering myeloma, asymptomatic MM, an symptomatic MM, ranging from newly diagnosed to late stage relapsed/refractory. Examples of lymphoma-related conditions include, e.g., Hodgkin's lymphoma or non-Hodgkin's lymphoma (NHL). In embodiments, the non-Hodgkin's Lymphoma is a selected from the group consisting of aggressive NHL, transformed NHL, indolent NHL, relapsed NHL, refractory NHL, low grade non-Hodgkin's Lymphoma, follicular lymphoma, large cell lymphoma, B-cell lymphoma, T-cell lymphoma, Mantle cell lymphoma, Burkitt's lymphoma. NK cell lymphoma, diffuse large B-cell lymphoma, acute lymphoblastic lymphoma, and cutaneous T cell cancer, including mycosos fungoides/Sezry syndrome.

In addition, the compositions disclosed herein can be used to treat or prevent an autoimmune disease, an inflammatory disease, an amyloid disease, a metabolic disorder, a developmental disorder, a cardiovascular disease, liver disease, an intestinal disease, an infectious disease, an endocrine disease and a neurological disorder. In embodiments a pharmaceutical composition to a subject that includes a polymeric nanoparticle comprising a block copolymer comprising poly(lactic acid) (PLA) and poly(ethylene glycol) (PEG) and bortezomib.

Inflammatory diseases include, e.g., multiple sclerosis (MS), systemic lupus erythematosus (SLE) fibrosis and antibody mediated rejection in transplantation, e.g. heart, lung, kidney or liver transplantation.

Amyloid diseases include, e.g., Alzheimer's disease, Lewy Body Dementia, Frontotemporal dementia, type 2 diabetes, Huntington's disease, Parkinson's disease, amyloidosis associated with hemodialysis for renal failure, Down syndrome, hereditary cerebral hemorrhage with amyloidosis, kuru, Creutzfeldt-Jakob disease, Gerstmann-Straussler-Scheinker disease, fatal familial insomnia, British familial dementia, Danish familial dementia, familial corneal amyloidosis, Familial corneal dystrophies, medullary thyroid carcinoma, insulinoma, isolated atrial amyloidosis, pituitary amyloidosis, aortic amyloidosis, plasma cell disorders, familial amyloidosis, senile cardiac amyloidosis, inflammation-associated amyloidosis, familial Mediterranean fever, systemic amyloidosis, and familial systemic amyloidosis) or a tauopathy (e.g., Frontotemporal dementia, chronic traumatic encephalopathy, progressive supranuclear palsy, corticobasal degeneration).

In an aspect, provided herein is a method for treating a disease in a subject in need thereof comprising administering to the subject a therapeutically effective amount of a pharmaceutical composition comprising a) a polymeric nanoparticle formed of a polymer comprising PLA-PEG di-block copolymer; and bortezomib.

In an embodiment of the methods provided herein, the pharmaceutical composition further comprises a chemotherapeutic agent or a targeted anti-cancer agent selected from the group consisting of lenalidomide, crizotinib, gleevec, herceptin, avastin, PD-1 checkpoint inhibitors, PDL-1 checkpoint inhibitors, CTLA-4 checkpoint inhibitors, doxorubicin, daunorubicin, decitabine, irinotecan, SN-38, cytarabine, docetaxel, triptolide, geldanamycin, 17-AAG, 5-FU, oxaliplatin, carboplatin, taxotere, methotrexate, paclitaxel, and an indenoisoquinoline.

In an embodiment of the methods provided herein, the disease is cancer, an autoimmune disease, an inflammatory disease, a metabolic disorder, a developmental disorder, a cardiovascular disease, liver disease, an intestinal disease, an infectious disease, an endocrine disease and a neurological disorder.

In another embodiment, the disease is cancer.

In yet another embodiment, the cancer is breast cancer, prostate cancer, non-small cell lung cancer, metastatic colon cancer, or pancreatic cancer.

In another embodiment, the cancer comprises a PD-1 refractory tumor.

In an embodiment of the methods provided herein, the nanoparticles are formed of a polymer consisting essentially of PLA-PEG di-block copolymer.

In an embodiment of the methods provided herein, the nanoparticles are formed of a polymer consisting essentially of PLA-PEG-PPG-PEG tetra-block copolymer.

In an embodiment, the polymeric nanoparticles are formed of a polymer consisting essentially of PLA-PEG di-block copolymer.

In an embodiment, the polymeric nanoparticles are formed of a polymer consisting essentially of PLA-PEG-PPG-PEG tetra-block copolymer.

As used herein, the term “administration” refers to the act of giving a drug, prodrug, antibody, or other agent comprising the polymeric nanoparticle to a physiological system (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, by injection (e.g., intravenously, subcutaneously, intratumorally, intraperitoneally, etc.) and the like.

In embodiments, the polymeric particles are administered intravenously (IV), subcutaneously (Sub-Cu) or intraperitoneally (IP).

The administration of a pharmaceutical composition provided herein may result not only in a beneficial effect with regard to alleviating, delaying progression of or inhibiting the symptoms, but also in further surprising beneficial effects, e.g. fewer side-effects, more durable response, an improved quality of life or a decreased morbidity, compared with, for example, delivering the agent without using the polymeric nanoparticle system described herein or by any other conventional means.

The effective dosage of the polymeric nanoparticles provided herein may vary depending on the particular protein, nucleic acid, and or other therapeutic agent used, the mode of administration, the condition being treated, and the severity of the condition being treated. Thus, the dosage regimen of the polymeric nanoparticle is selected in accordance with a variety of factors including the route of administration and the renal and hepatic function of the patient.

To determine efficacy, treatment may further comprise comparing one or more pre-treatment or post-treatment phenotypes to a standard phenotype. The standard phenotype is the corresponding phenotype in a reference cell or population of cells. Reference cells are one or more of the following, cells from a person or subject that is not suspected of having a protein degradation disorder, cells from the subject, cultured cells, cultured cells from the subject, or cells from the subject pre-treatment. Cells from the subject may include, for example, a bone marrow stromal cell, (BMSC), a peripheral blood mononuclear cell (PBMC), lymphocytes, hair follicles, blood cells, other epithelial cells, bone marrow plasma cells, primary cancer cells, patient derived tumor cells, normal or cancerous hematopoietic stem cells, neural stem cells, solid tumor cells, astrocytes, and the like.

### Combination Treatments

The compositions provided herein optionally further comprise an additional treatment modality, e.g., a therapeutic agent (e.g., a chemotherapeutic agent), radiation agent, hormonal agent, biological agent or an anti-inflammatory agent that is administered to a subject along with bortezomib.

Therapeutic agents that can be used in a combination therapy with bortezomib may include, e.g, lenalidomide, crizotinib or a histone deacetylase inhibitor (HDAC), such as those disclosed in U.S. Pat. No. 8,883,842. Additional therapeutic agents include, e.g., gleevec, herceptin, avastin, PD-1 checkpoint inhibitors, PDL-1 checkpoint inhibitors, CTLA-4 checkpoint inhibitors, tamoxifen, trastuzamab, raloxifene, doxorubicin, fluorouracil/5-fu, pamidronate disodium, anastrozole, exemestane, cyclophos-phamide, epirubicin, letrozole, toremifene, fulvestrant, fluoxymester-one, trastuzumab, methotrexate, megastrol acetate, docetaxel, paclitaxel, testolactone, aziridine, vinblastine, capecitabine, goselerin acetate, zoledronic acid, taxol, vinblastine, and/or vincristine. Useful non-steroidal anti-inflammatory agents, include, but are not limited to, aspirin, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam, isoxicam; salicylic acid derivatives, including aspirin, sodium salicylate, choline magnesium trisalicylate, salsalate, diflunisal, salicylsalicylic acid, sulfasalazine, and olsalazin; para-aminophennol derivatives including acetaminophen and phenacetin; indole and indene acetic acids, including indomethacin, sulindac, and etodolac; heteroaryl acetic acids, including tolmetin, diclofenac, and ketorolac; anthranilic acids (fenamates), including mefenamic acid, and meclofenamic acid; enolic acids, including oxicams (piroxicam, tenoxicam), and pyrazolidinediones (phenylbutazone, oxyphenthartazone); and alkanones, including nabumetone and pharmaceutically acceptable salts thereof and mixtures thereof. For a more detailed description of the NSAIDs, see Paul A. Insel, Analgesic-Antipyretic and Antiinflammatory Agents and Drugs Employed in the Treatment of Gout, in Goodman & Gilman's The Pharmacological Basis of Therapeutics 617-57 (Perry B. Molinhoff and Raymond W. Ruddon eds., 9.sup.th ed 1996) and Glen R. Hanson, Analgesic, Antipyretic and Anti-Inflammatory Drugs in Remington: The Science and Practice of Pharmacy Vol II 1196-1221 (A. R. Gennaro ed. 19th ed. 1995) which are hereby incorporated by reference in their entireties.

In an embodiment, the additional chemotherapeutic agent or a targeted anti-cancer agent selected from the group consisting of doxorubicin, daunorubicin, decitabine, irinotecan, SN-38, cytarabine, docetaxel, triptolide, geldanamycin, 17-AAG, 5-FU, oxaliplatin, carboplatin, taxotere, methotrexate, paclitaxel, and an indenoisoquinoline. Although the subject matter has been described in considerable detail with reference to certain embodiments thereof, other embodiments are possible. As such, the spirit and scope of the appended claims should not be limited to the description of the specific embodiments contained therein.

### Examples

The disclosure will now be illustrated with working examples, and which is intended to illustrate the working of disclosure and not intended to restrictively any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

### Example 1. Preparation of Polymeric Nanoparticles of PLA-PEG-PPG-PEG Block Copolymer

Poly(lactic acid) (MW.—45,000-60,000 g/mol), PEG-PPG-PEG (Table 1) and tissue culture reagents were obtained from Sigma-Aldrich (St. Louis, Mo.). All reagents were analytical grade or above and used as received, unless otherwise stated. Cell lines were obtained from NCCS Pune, India or from ATCC, Maryland, USA

5 gm of poly (lactic acid) (PLA) with an average molecular weight of 60,000 g/mol was dissolved in 100 ml CH₂Cl₂ (dichloromethane) in a 250 ml round bottom flask. To this solution, 0.7 g of PEG-PPG-PEG polymer (molecular weight range of 1100-8400 Mn) was added. The solution was stirred for 10-12 hours at 0° C. To this reaction mixture, 5 ml of 1% N,N-dicyclohexylcarbodimide (DCC) solution was added followed by slow addition of 5 ml of 0.1% 4-Dimethylaminopyridine (DMAP) at −4° C. to 0° C./subzero temperatures. The reaction mixture was stirred for the next 24 hours followed by precipitation of the PLA-PEG-PPG-PEG block copolymer with diethyl ether and filtration using Whatman filter paper No. 1. The PLA-PEG-PPG-PEG block copolymer precipitates so obtained were dried under low vacuum and stored at 2° C. to 8° C. until further use.

The PLA-PEG-PPG-PEG nanoparticles were prepared by an emulsion precipitation method. 100 mg of the PLA-PEG-PPG-PEG copolymer obtained by the above-mentioned process was separately dissolved in an organic solvent, for example, acetonitrile, dimethyl formamide (DMF) or dichloromethane to obtain a polymeric solution.

The nanoparticles were prepared by adding this polymeric solution drop wise to the aqueous phase of 20 ml distilled water. The solution was stirred magnetically at room temperature for 10 to 12 hours to allow residual solvent evaporation and stabilization of the nanoparticles. The nanoparticles were then collected by centrifugation at 25,000 rpm for 10 min and washed thrice using distilled water. The nanoparticles were further lyophilized and stored at 2° C. to 8° C. until further use.

The shape of the nanoparticles obtained by the process mentioned above is essentially spherical. The particle size range was about 30 to 120 nm. The hydrodynamic radius of the nanoparticle was measured using a dynamic light scattering (DLS) instrument and is in the range of 110-120 nm.

### Example 2. Preparation of a Bortezomib-Encapsulated Nanoparticle

The nanoparticles of the present invention are amphiphilic in nature and are capable of being loaded with both hydrophobic drugs like bortezomib.

100 g of the PLA-PEG-PPG-PEG nanoparticle prepared using the process of Example 1 was dissolved in 5 ml of an organic solvent like acetonitrile (CH₃CN), dimethyl formamide (DMF; C₃H₇NO), acetone or dichloromethane (CH₂Cl₂).

1-5 mg of bortezomib was dissolved in an aqueous solution and is added to the above polymeric solution. Bortezomib is usually taken in the weight range of about 10-20% weight of the polymer. This solution is briefly sonicated for 10-15 seconds at 250-400 rpm produce a fine primary emulsion.

The fine primary emulsion is added drop wise using a syringe/micropipette to the aqueous phase of 20 ml distilled water and stirred magnetically at 250 to 400 rpm at 25° C. to 30° C. for 10 to 12 h in order to allow solvent evaporation and nanoparticle stabilization. The aqueous phase further comprises a sugar additive. The resulting nanoparticle suspension was allowed to stir overnight, in an open, uncovered condition to evaporate the residual organic solvent. The bortezomib encapsulated polymeric nanoparticles were collected by centrifugation at 10,000 g for 10 min or by ultrafiltration at 3000 g for 15 min. (Amicon Ultra, Ultracel membrane with 100,000 NMWL, Millipore, USA). The nanoparticles were resuspended in distilled water, washed thrice and lyophilized. They were stored at 2° C. to 8° C. until further use. The polymeric nanoparticles were highly stable. Comparison of the loading efficacy of the polymeric nanoparticle prepared using different weights of the co-polymer.

### Example 3. Effect of Bortezomib-Containing Nanoparticles on Cell Proliferation/Viability of Multiple Myeloma Cell Lines RPMI-8226 and OPOM-2

The effect of bortezomib-containing nanoparticles on multiple myeloma cell viability was assessed using Alamar Blue reagent. Based on their growth rate, 1500 to 4000 cells/well RPMI-8226 and OPOM-2 were plated in 96 well plates and allowed to grow overnight at 37° C., 5% CO2. Cells were treated with different concentrations of bortezomib-containing nanoparticles for five days with three-fold serial dilutions for eight concentrations.

Alamar Blue reagent (1:10 dilution in the culture medium) was then added to the wells and incubated for 2-4 hrs. The change in absorption was measured with excitation at 570 nM and emission at 600 nM. The percentage viability was calculated compared to the untreated control as 100%. The dose response curves were plotted using two different soft-wares (left and right).

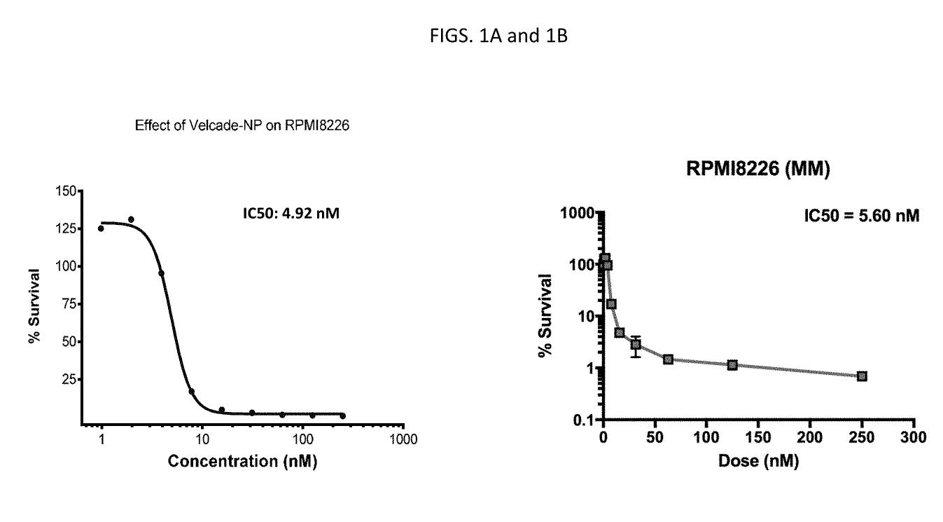

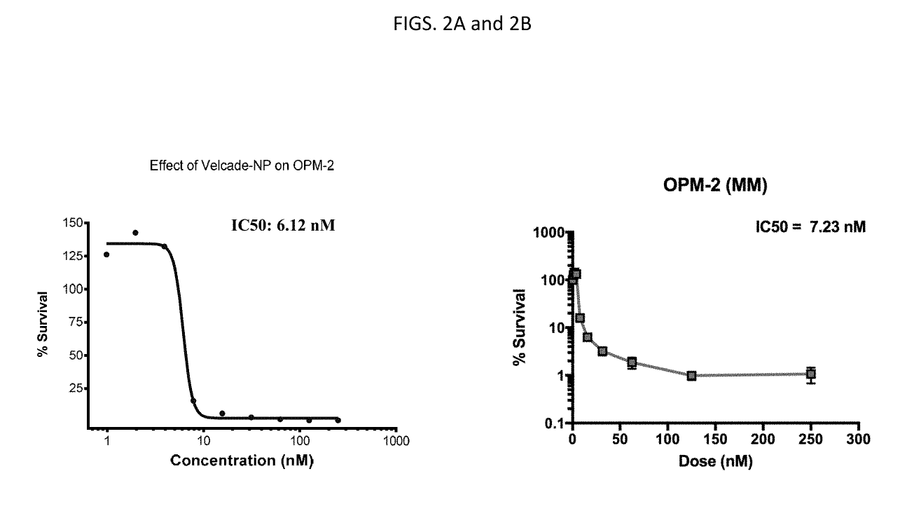

The results are shown in FIG. 1 (RPMI-8226) and FIG. 2 (OPOM-2). Shown in both graphs is the percent survival (y-axis) as a function of nanoparticle concentration (x-axis).

For the RPMI-8226 cell line the IC50 was 4.92 nM and 5.6 nM (FIG. 1). For the OPOM-2 cell line the IC50 was 6.12 nM and 7.23 nM (FIG. 2). The values were comparable in the two cell lines.

### Example 4: Assessment of Antitumor Activity of Bortezomib-Containing Nanoparticles Against RPMI-8226 Cells Implanted in Mice

The ability of bortezomib-containing nanoparticles to inhibit growth of RPMI-822 tumor cells implanted in mice was examined.

4 to 6-week-old Balb/c nu/nu mice were injected subcutaneously with 5×10⁶ RPMI-8226 multiple myeloma cells in the left flank. Mice with established RPMI-8226 tumors (90-120 mm³) were randomized into groups of 6 mice each and treated i.p. (i) each day with vehicle control or (ii) once each week with 1 mg/kg VEL-nanoparticles for 3 weeks. Tumors were measured every other day with calipers, and tumor volumes were calculated using the formula (AXB²)/0.5, where A and B are the longest and shortest tumor diameters, respectively. Statistical analysis of tumor volumes was performed by one-way ANOVA and the Dunnett test using Origin 8.0 (Origin Lab).

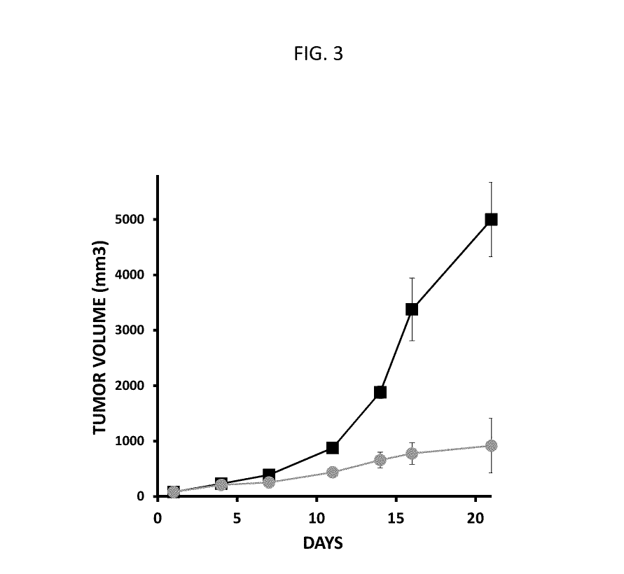

The results are shown in FIG. 3. Shown is tumor volume (y-axis) over time (x-axis). Tumor volume in mice treated with vehicle control reached 5000 mm³. In contrast, tumor volume in mice treated with bortezomib-containing nanoparticles did not exceed 1000 mm³.

### Example 5. Assessment of Body Weight in RPMI-8226 Multiple Myeloma (MM) Xenograft Mice Treated with Bortezomib-Containing Nanoparticles

The body weight in RPMI-8226 MM xenograft mice treated with bortezomib-containing nanoparticles and control mice as discussed in Example 2 above was examined for 21 days and compared to body weight over the same time in mice treated with vehicle.

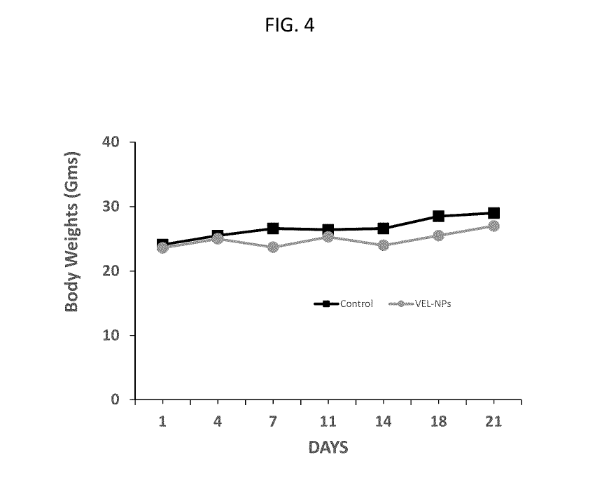

The results are shown in FIG. 4. Body weight remained stable or slightly increased in both groups during the length of the study. These results demonstrate that the bortezomib-containing nanoparticles do not adversely affect body weight.

### Example 6. Comparative Toxicity in Wild-Type Mice of Varying Doses of Bortezomib and Bortezomib-Containing Nanoparticles

The effect of nanoparticles in mitigating the toxicity of bortezomib was examined in wild-type mice. Different doses of bortezomib alone (1.5, 3, 6, 9 and 12 mg/kg) or bortezomib-nanoparticles (NP) (0.9, 1.8, 3.6, 5.4 and 7.2 mg/kg) were injected into CD1 wild type. Three mice were used in each group of bortezomib alone and bortezomib-NP groups, body weights, food and water uptake were measured every day for 22 days.

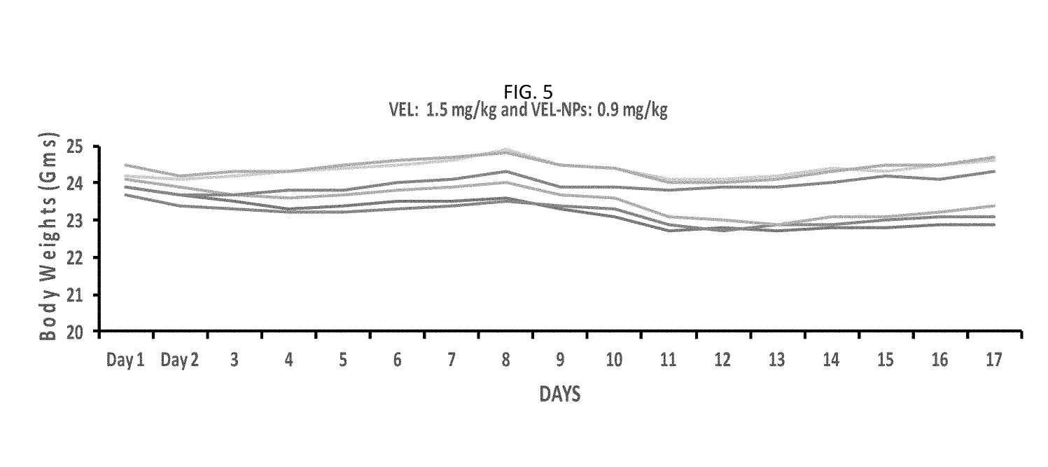

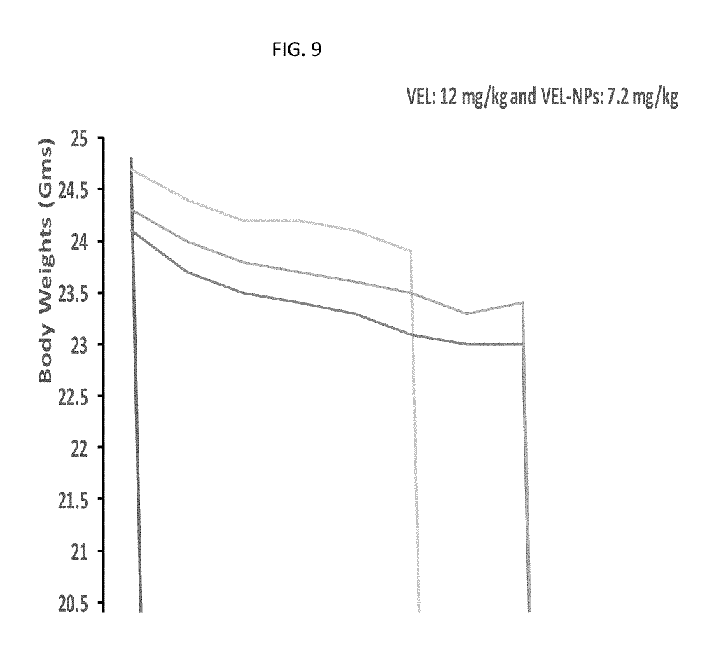

The results are shown in FIGS. 5-9. The results show body weight changes or lethality in mice treated with bortezomib alone and bortezomib-NPs. At the lowest dose of bortezomib tested body weight was significantly higher at the end of the study in mice treated with bortezomib-containing nanoparticles relative to mice treated with bortezomib alone (FIG. 5; 1.5 mg/kg dose of bortezomib and 0.9 mg/kg dose of bortezomib-NPs).

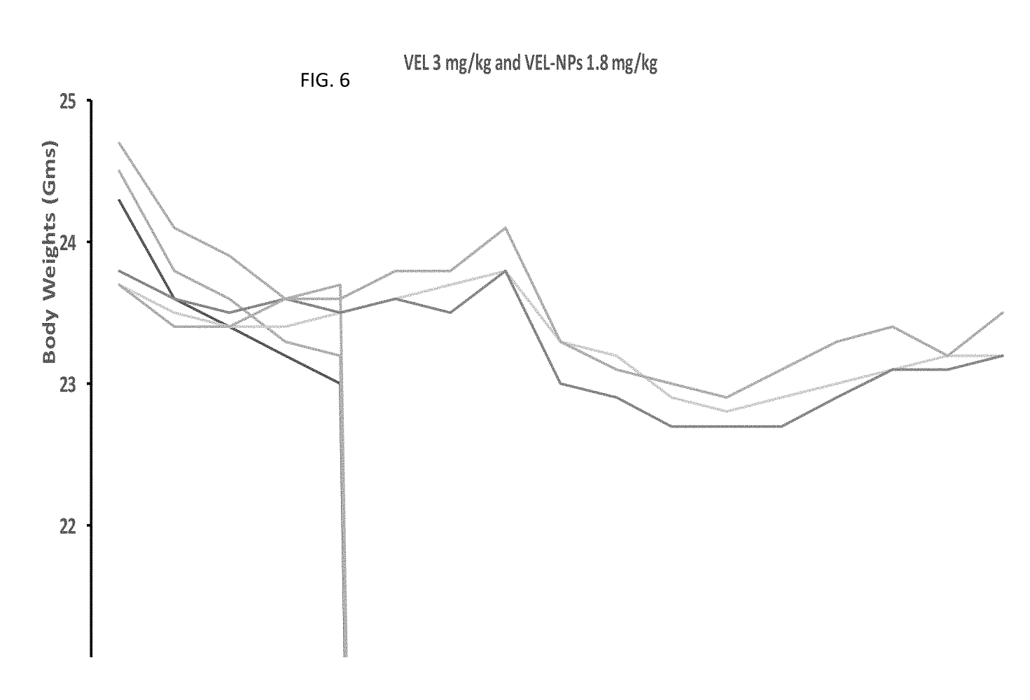

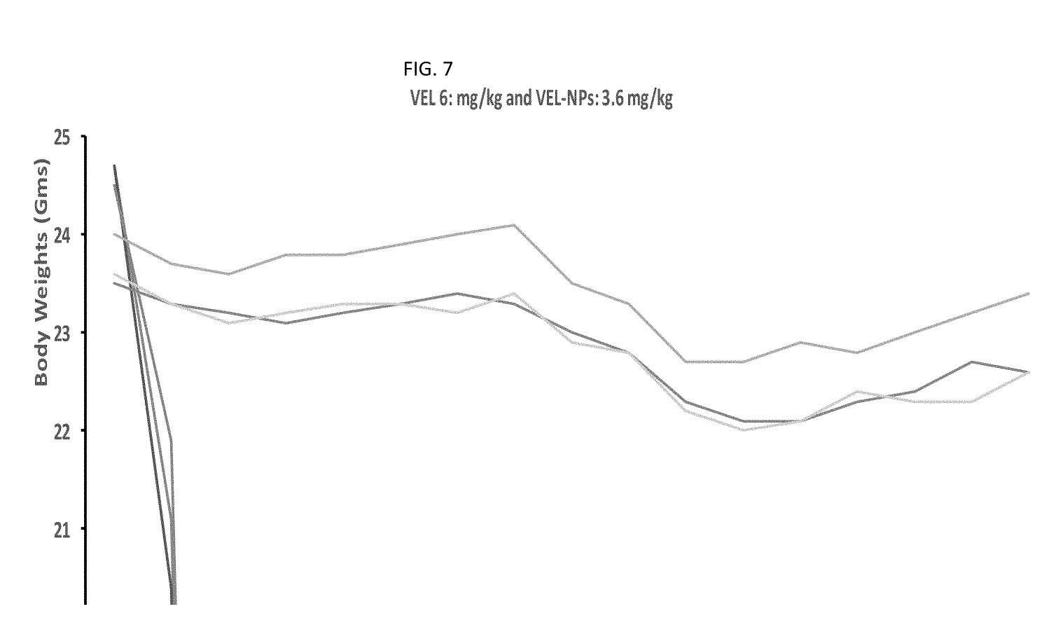

At the next two higher doses tested, lethality was observed tested in mice treated with bortezomib alone; no mouse treated with bortezomib alone survived longer than five days (FIG. 6; 3 mg/kg dose) or two days ((FIG. 7; 6 mg/kg dose). In contrast, mice treated with bortezomib-containing nanoparticles at 1.8 and 3.6 mg/kg concentrations survived for the duration of the study with body weight essentially unchanged.

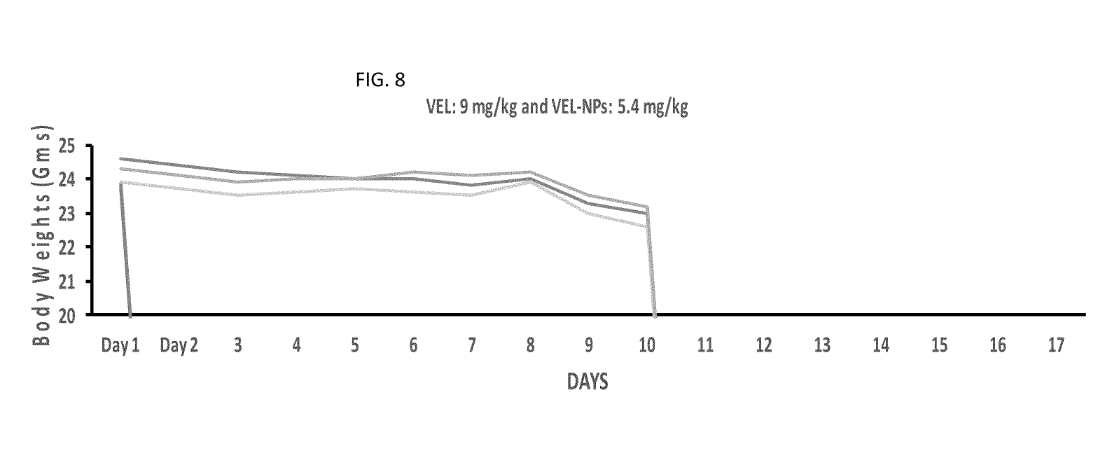

Lethality was observed in both groups at the highest concentration of bortezomib tested (FIG. 8; 9 mg/kg). However, mice in the bortezomib-containing nanoparticle group survived until Day 10 of the study at 5.4 mg/kg dose, while all members of the group treated with bortezomib in the absence of nanoparticles died after Day 1.

These results demonstrate that nanoparticles mitigate the toxic effects of increasing doses of bortezomib in mice.

### Example 7. Characterization of Bortezomib-Containing Nanoparticles

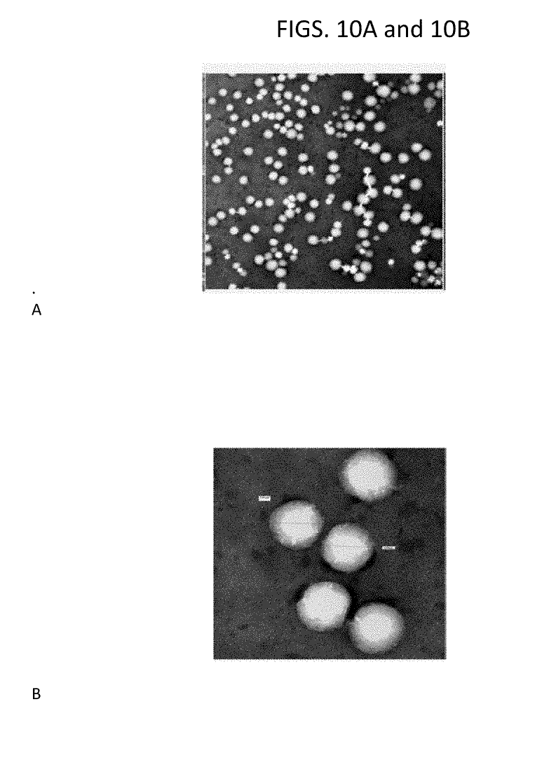

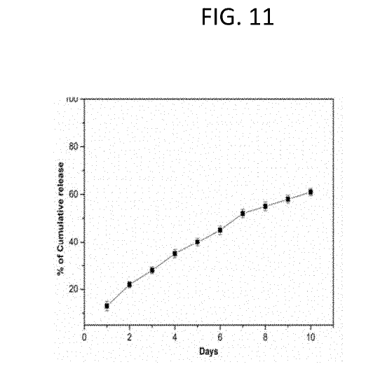

FIGS. 10A and 10B provide transmission electron micrographs providing the size and shape of the bortezomib containing nanoparticles used in the Examples above. The diameter shown by red line in two NPs in FIG. 10B is 130 nm. FIG. 11 is a graph showing the slow and sustained release of bortezomib from the nanoparticles over 10 days in an in vitro cell free buffer system.

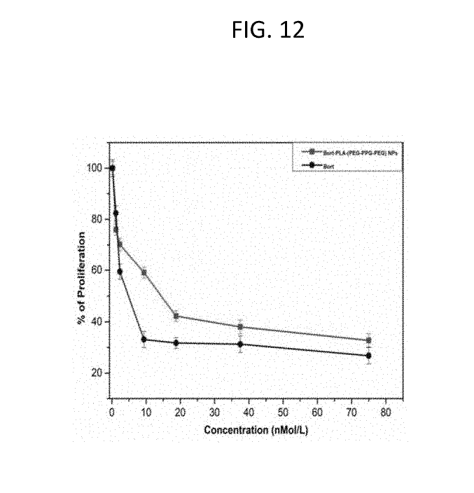

FIG. 12 provides preliminary initial data showing that bortezomib-nanoparticles reduce proliferation of MCF-7 hormone-dependent breast cancer cell line. MCF-7 breast cancer cells were treated with different concentrations of bortezomib (Blue curve on the bottom) or bortezomib-NPs (red curve on the top) for 48 hours. Cell proliferation was measured by trypan blue dye exclusion. The IC50 of bortezomib-NPs is <20 nM.

### Example 8. Comparison of In Vivo Efficacy of VEL-NPs by in Different Routes of Administration

To investigate whether VEL-NPs are effective in different routes of administration, in vivo studies were performed in nu/nu mice bearing established subcutaneous RPMI-8226 multiple myeloma tumors. Based on the kinetics of bortezomib release from NPs over 7 days, mice bearing RPMI-8226 multiple myeloma tumors, intraperitoneally (i.p.) 1 mg/kg once a week for 3 weeks.

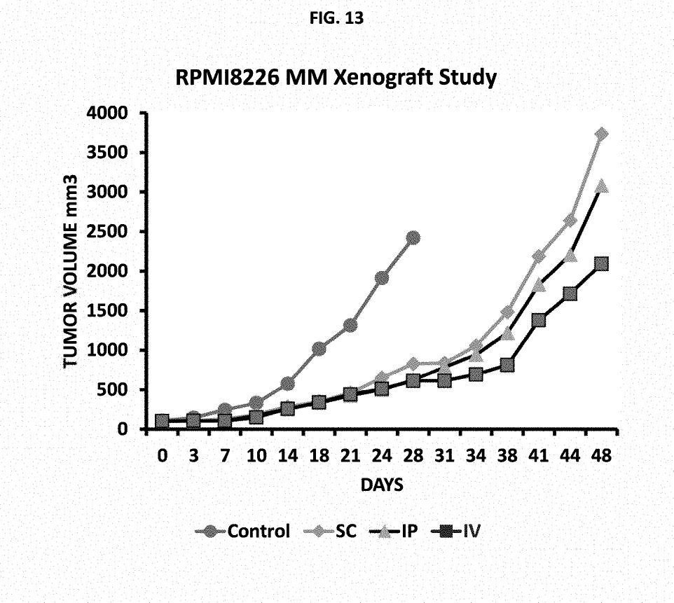

The results are presented in FIG. 13, which is a graph showing tumor volume (mm³) over time of RPMI-8226 multiple myeloma cells grown as s.c. xenograft in nu/nu mice administered SC (diamond symbols), IP (triangle symbols) or IV (square symbols). Control mice receiving NPs lacking bortezomib (“empty NPs”) are denoted with circular symbols. As compared with mice treated with nanoparticles not including bortezomib (empty NPs), treatment with 1 mg/kg VEL-NPs was associated with substantial regression of the tumors (FIG. 13). Interestingly, treatment of mice with 1 mg/kg VEL-NPs either intravenously (IV), subcutaneously (Sub-Cu) or intra-peritoneally (IP) was associated with similar regression of the tumors (FIG. 13).

Analysis of survival further demonstrated that mice treated with VEL-NPs with all the three routes of administration survived significantly longer than those treated with empty NPs. Significantly, there was no weight loss or other overt toxicities observed in mice treated with VEL-NPs (data not shown). Extensive tissue necrosis was observed in the VEL-NPs-treated group.

We claim:

1. A composition comprising
a) polymeric nanoparticles comprising a poly(lactic acid)-poly(ethylene glycol)-poly(propylene glycol)-poly(ethylene glycol) (PLA-PEG-PPG-PEG) tetra block copolymer, and
b) bortezomib.

2. The composition of claim 1, wherein the PLA-PEG-PPG-PEG tetra-block copolymer is formed from chemical conjugation of PEG-PPG-PEG tri-block copolymer with PLA.

3. The composition of claim 1, wherein the molecular weight of PLA is between about 10,000 and about 100,000 Daltons.

4. The composition of claim 1, wherein the molecular weight of PLA is between about 20,000 and 90,000 Daltons.

5. The composition of claim 1, wherein the molecular weight of PLA is between about 30,000 and 80,000 Daltons.

6. The composition of claim 1, wherein the molecular weight of PEG-PPG-PEG is between about 8,000 Daltons and 18,000 Daltons.

7. The composition of claim 1, wherein the molecular weight of PEG-PPG-PEG is between about 10,000 Daltons and 15,000 Daltons.

8. The composition of claim 1, wherein the molecular weight of PLA in the copolymer is between 17,000 Daltons and 72,000 Daltons and the molecular weight of PEG-PPG-PEG is 12,500 Daltons.

9. The composition of claim 1, further comprising a second therapeutic agent or a targeted anti-cancer agent.

10. The composition of claim 9, wherein the second therapeutic agent is selected from the group consisting of crizotinib, lenalidomide, gleevec, herceptin, avastin, PD-1 checkpoint inhibitors, PDL-1 checkpoint inhibitors, and CTLA-4 checkpoint inhibitors.

11. A pharmaceutical composition comprising the composition of claim 1 and a pharmaceutically acceptable carrier.

12. The pharmaceutical composition of claim 11, wherein the polymeric nanoparticle further comprises a targeting moiety attached to the outside of the polymeric nanoparticles.

13. A method of treating a cell exhibiting symptoms of cancer comprising contacting the cell with a therapeutically effective amount of the compound of claim 1.

14. The method of claim 13, wherein the cell is one or more of a cell from a subject or a cultured cell.

15. The method of claim 14, wherein the cell from the subject is one or more of bone marrow stromal cell (BMSC), a peripheral blood mononuclear cell (PBMC), lymphocytes, hair follicles, blood cells, other epithelial cells, bone marrow plasma cells, primary cancer cells, patient derived tumor cells, normal or cancerous hematopoietic stem cells, neural stem cells, solid tumor cells, or astrocytes.

16. A method for treating a subject at risk for or having a hematological malignancy or disorder associated with same, the method comprising administering to a subject in need thereof a therapeutically effective amount of the compound of claim 1 and a pharmaceutically effective carrier.

17. The method of claim 16, wherein the hematological malignancy or disorder is multiple myeloma (MM) or lymphoma.

18. The method of claim 16, wherein the hematological malignancy is myelodysplastic syndrome, Hodgkin's lymphoma, chronic lymphocytic leukemia, acute myelogenous leukemia or B cell lymphoma.

19. The method of claim 17, wherein the subject is at risk for monoclonal Gammopathy of Undetermined Significance (MGUS), smoldering myeloma, asymptomatic MM, or symptomatic MM.

20. The method of claim 19, wherein the symptomatic MM is newly diagnosed MM.

21. The method of claim 19, wherein the symptomatic MM is late stage relapsed/refractory MM.

22. The method of claim 16, further comprising administering an additional anti-cancer therapy to the subject.

23. The method of claim 22, wherein the additional anti-cancer therapy is surgery, chemotherapy, radiation, hormone therapy, immunotherapy, or a combination thereof.

24. The method of claim 22, wherein the additional anti-cancer therapy reduces bone absorption.

25. The method of claim 22, wherein the additional anti-cancer therapy reduces osteoclast mediated bone resorption.

26. The method of claim 24, wherein the additional anti-cancer therapy is a bisphosphonate.

26. The method of claim 17, wherein the subject is a human.

27. The method of claim 17, wherein administration is via a route selected from the group consisting of subcutaneous, intravenous, and intraperitoneal delivery.

28. The method of claim 17, wherein administration of the composition does not induce weight loss in the subject.

29. A method of reducing proliferation, survival, migration, or colony formation ability of multiple myeloma cells in a subject with multiple myeloma, the method comprising administering to the subject a therapeutically effective amount of the compound of claim 1 and a pharmaceutically effective carrier.

30. The method of claim 29, wherein administration is via a route selected from the group consisting of subcutaneous, intravenous, and intraperitoneal delivery.

31. A method of inhibiting metastasis of myeloma in a subject, the method comprising administering to a subject with myeloma a therapeutically effective amount of the composition of claim 1 and a pharmaceutically effective carrier.

32. The method of claim 31, wherein administration is via a route selected from the group consisting of subcutaneous, intravenous, and intraperitoneal delivery.
